(19) United States
(12) Patent Application Publication
(10) Pub. No.: US 2002/0019631 A1
(43) Pub. Date: Feb. 14, 2002

(54) Electro-surgical pencil with smoke evacuation

(76) Inventors: John Kidder, Vancouver (CA); Volf Smus, Richmond (CA); Douglas Broadfoot, Delta (CA); Sherrill Moreno, Trail (CA)

Correspondence Address: Hall, Priddy, Myers & Vande Sande, Suite 200, 10220 River Road, Potomac, MD, 20854, US

(21) Appl. No.: 09/783,128

(22) Filed: Feb. 15, 2001

(30) Foreign Application Priority Data
Feb. 28, 2000 (CA) 2,299,752

(51) Int. Cl. A61B 18/14

(52) U.S. Cl. 606/42; 606/45; 604/35

(57) ABSTRACT

According to the invention there is provided an electro-surgical pencil of a type having an electrode projecting out one end of said pencil, an electrical coupling operative to couple the electrode to an external electrical source. The pencil has an elongated housing having a hollow interior passageway, an open electrode end through which the electrode projects and an open end opposite to the electrode end couplable to a vacuum source. The hollow interior passageway is of a diameter sufficiently large to permit a flow and velocity of air sufficient to aspirate and evacuate smoke produced by the pencil. An electrode support in the hollow interior passageway holds the electrode. The electrode support and the electrode present a cross-sectional area which is substantially less than that of the hollow interior passageway. An optical switch is coupled to the elongated housing. An optical reflector having at least two different types of reflective surfaces is also coupled to the elongated housing proximate the optical switch, and a light fiber is coupled to the switch. An end of the light fiber is proximate the reflector, and is positionable to reflect light selectably from one of the two different types of reflective surfaces back up the light fiber.

### FIELD

The invention relates in general to electro-surgical pencils. More particularly, but not exclusively, the present invention relates to an electro-surgical pencil with smoke evacuation.

### BACKGROUND

Many modern surgical procedures necessitate the delivery of energy into tissue by either electro-cautery or by surgical laser. In some procedures tissue is precisely vaporized producing a clean cut. In other procedures tissue is heated to coagulate cells or charred to cauterize tissue and prevent bleeding. In all cases precise control by the surgeon is important for safe and effective treatment. Much effort has been and continues to be expended to improve the ergonomic design and operating features of surgical hand pieces that deliver this energy.

Surgical smoke is usually the result of surgical procedures, such as cutting and coagulation of human tissue with a laser or electro surgical device. Besides having an unpleasant and strong odour, the resulting smoke includes water vapors, various organic gases and vapors, particles and may include pathogens such as hepatitis or human immunodeficiency virus (HIV) viral particles. Some of the smoke components are believed to be carcinogenic. Facemasks and face shields offer a limited protection for the medical personnel. The presence of smoke in the surgical field can also obscure the surgeon's view of the procedure being performed, which can compromise accuracy and safety. There is, without question, a need for safely removing smoke from the surgical field. There is an associated need for doing so in a manner that does not compromise ergonomic performance.

Devices for removing smoke, tissue and fluids from the surgical field are known in the art and many designs have been implemented. Prior devices have used a variety of different surgical tools and suction combinations. See, for example, U.S. Pat. Nos. 3,974,833, 3,902,494 and 3,828,780. In U.S. Pat. No. 3,974,833, a disposable electrical surgical cautery with suction control feature is shown wherein suction and power are supplied separately to a single non-interchangeable tool. In U.S. Pat. No. 3,902,494, a suction surgical instrument is shown having an electrode near the suction tube to prevent clogging of the suction port. U.S. Pat. No. 3,828,780 discloses an electro-coagulator suction instrument having an open-ended metal tube that is inserted in an instrument for contact with the suction passageway and an exposed wire is provided. Separate suction and electrical lines supply suction and power to the instrument.

U.S. Pat. No. 4,347,842 discloses an expandable, electro-surgical suction tube and instrument holder for use in microsurgery that has a receptacle in one end that provides both power and suction to a variety of surgical instruments that can be connected thereto.

U.S. Pat. No. 3,982,541 discloses a surgical device having a central tube through which a surgical laser can be directed and an outer tube that can be attached to a means to remove smoke and vaporized tissue from the site of surgery.

All of the above devices incorporate some type of chamber in the surgical hand-piece through which surgical smoke can be removed. The use of such a chamber adds to the size of the hand-piece rendering it more cumbersome and awkward to use than devices without smoke evacuation. There is a need for a surgical hand-piece that can accomplish smoke evacuation while minimizing reduction of ergonomic performance.

Most electro-surgical devices are actuated by a hand control or foot control electrically connected to the surgical generator to control the application and type of energy delivered to the surgical hand-piece. The high voltages directed to the active electrode require that the control and active electrode wires have significant dielectric isolation. This often causes the electrical cable between the hand-piece and the surgical generator to be stiffer than desirable for good ergonomics. There is a need for improvements to the cable connecting the surgical hand-piece that can increase flexibility and minimize resistance to the surgeon's hand movements.

The addition of a tube connecting to the hand-piece for evacuating smoke from the surgical region to the smoke evacuation device further compromises the ergonomic performance of the hand-piece. This extra tubing adds to the mass and provides additional resistance to the surgeon's hand movements. There is a need for improvements to the tubing and/or connection methods to minimize this resistance.

Smoke evacuation devices can be continuously applied during surgery but the noise of the suction and filtration systems is often annoying to clinical staff. Attempts have been made to mitigate this by providing manual or automatic control systems to activate the smoke suction device only as needed. U.S. Pat. No. 5,160,334 discloses an electro-surgical generator and suction apparatus incorporating switching circuitry to simultaneously activate the smoke removal module when the electro-surgical tool is activated. U.S. Pat. No. 5,318,516 discloses an automatic smoke evacuator system incorporating sensors that detect the presence of the radio frequency field generated when the surgical laser or electro-surgical apparatus is activated and activate switching circuitry to simultaneously activate the smoke removal device. While these inventions address the need for manual or automated control of smoke evacuation, they do not address the overall need for improved ergonomics. Additionally they must be incorporated into the design of the surgical generator, increasing cost and requiring expensive replacement of existing surgical devices.

U.S. Pat. No. 5,853,410 discloses an invention that provides for a device that performs a similar function to that of U.S. Pat. No. 5,318,516, except that it can be retrofitted to an existing generator. While reducing the costs associated with equipment replacement the latter device still does not address the overall ergonomic issues of size and cumbersomeness associated with electrically controlled electro-surgical or laser surgical hand-pieces.

As can be seen from the above, despite the extended use of electro-surgical pencils, they still suffer from drawbacks, which are seemingly inherent to their basic engineering concepts.

There is accordingly a need for an electro-surgical pencil for use with smoke evacuation, which obviates the disadvantages of the prior art.

### SUMMARY OF THE INVENTION

Accordingly it is an object of the present invention to develop an improved electro-surgical pencil, which is adapted for use with smoke evacuation.

It is a further object of the present invention to develop an electro-surgical pencil which does not require the use of electrically powered conductors in the pencil housing and thereby avoid the various potential modes of electrical failure when such conductors are exposed to certain liquids, such as biological fluids, to eliminate the potential for explosions in an explosive environment and to achieve a reduction in the requirement for electrical and environmental insulation and hence produce a more ergonomically effective device.

It is yet another object of the present invention to develop an electro-surgical pencil with smoke evacuation wherein the smoke is aspired as needed, unobtrusively and in a relatively unobstructed manner.

One method of addressing the ergonomic deficiencies of electrically switched devices is to replace the electrical control wires and associated thick and stiff electrical insulation with optical fiber and optical control systems that do not require dielectric insulation. U.S. Pat. No. 5,892,862, issued to Kidder et al., describes a fiber optic switching system that includes an optical switch having a movable actuator and a light fiber coupled to the actuator. Light directed into the fiber contacts a flexible reflective film whose reflectivity is conditioned to provide at least two different reflective surfaces. The fiber in the actuator abuts or is placed in close proximity to the film throughout its movement from one position to another. A detector detects light reflected from the film. The actuator is movable so as to direct light from the light fiber from the one reflective surface of the film to another and the detector detects light reflected from the film so as to determine which reflective surface of the film from which light has been reflected. By employing such a device in an electro-surgical pencil or similar device one can utilize the small size of the light fiber to achieve an overall smaller, more flexible mechanical assembly in combination with its other advantages.

While such switching systems have been used for some time, they have not been applied to electro-surgical or laser surgical devices with smoke evacuation capability, nor have they been adapted to control the activation of the smoke evacuation device.

According to the invention there is provided an electro-surgical pencil of a type having an electrode projecting out one end of said pencil, an electrical coupling operative to couple the electrode to an external electrical source. The pencil has an elongated housing having a hollow interior passageway, an open electrode end through which the electrode projects and an open end opposite to the electrode end couplable to a vacuum source. The hollow interior passageway is of a diameter sufficiently large to permit a flow and velocity of air sufficient to aspirate and evacuate smoke produced by the pencil. An electrode support in the hollow interior passageway holds the electrode. The electrode support and the electrode present a cross-sectional area, which is substantially less than that of the hollow interior passageway. An optical switch is coupled to the elongated housing. An optical reflector having at least two different types of reflective surfaces is also coupled to the elongated housing proximate the optical switch; and a light fiber is coupled to the switch. An end of the light fiber is proximate the reflector, and is positionable to reflect light selectably from one of the two different types of reflective surfaces back up the light fiber.

A switch sensor and a beam splitter may be coupled to the light fiber and be operative to separate reflected light from transmitted light and to direct the reflected light to the switch position sensor.

The vacuum source is coupled to the switch position sensor and is operative to become activated in response to the switch sensor detecting the optical switch being switched to activate the electrode.

Preferably, the hollow interior passageway is circular.

The switch may occupy less than 20% of the cross sectional area of the hollow interior passageway.

The light fiber may extend along the hollow interior passageway and through an external hollow coupling to the vacuum source.

The fiber optic switch may be at least partially disposed in the interior of the elongated housing means.

The elongated housing may have a substantially constant cross-section.

The elongated housing may have a ribbed zone on the exterior of the distal extremity.

In another aspect of the invention there is provided an electro-surgical pencil of a type having an electrode at a proximate extremity of the pencil, an electrical conductor coupled to the electrode and operative to couple said electrode to an external source of electrical power. The electro-surgical pencil has an elongated housing with a hollow interior passageway, and a distal extremity opposite to the proximate extremity couplable to a vacuum source. The hollow interior passageway is of a diameter sufficiently large to permit a flow and velocity of air sufficient to aspirate and evacuate smoke produced by the pencil. An optical switch is coupled to the elongated housing and is operative to switch power to the electrode in response to movement of the optical switch to a selected switch position. A ball socket joint is coupled to the distal extremity.

The electro-surgical pencil may include an electrode support in the hollow interior passageway operative to hold the electrode. The electrode support and the electrode advantageously may present a cross-sectional area, which is substantially less than that of the hollow interior passageway.

The ball and socket joint may have a ball and a socket, with the socket having indents around its periphery to provide flexibility for insertion of the ball.

The ball and socket may each be shells having a hollow interior.

Preferably, the ball and socket have tubular portions affixed to said ball and said socket, respectively, and a diameter of each of said ball and said socket are larger than a diameter of their respective tubular portions.

The tubular portion of one of said ball and socket may have spaced apart circumferential ribs operative to grip a tube inserted thereover.

### BRIEF DESCRIPTION OF THE DRAWINGS

Although the characteristic features of the invention will be particularly pointed out in the claims, the invention itself, and the manner in which it may be made and used, may be better understood by referring to the following description taken in connection with the accompanying drawings forming part thereof, wherein like reference numerals refer to like parts:

FIG. 1 shows a side-sectional elevation view of the electro-surgical pencil with an attached light fiber cable;

FIG. 2 shows a side-sectional elevation view of the top half of the elongated tubular housing of the electro-surgical pencil;

FIG. 3 shows the assembly comprising the light fiber cable, the fiber optic switch and the electrode blade;

FIG. 4 shows a side-sectional elevation view of the bottom half of the elongated tubular housing;

FIG. 5 shows an enlarged view of the switch and associated area of FIG. 1;

FIG. 6 shows a side-sectioned elevation view of an electro-surgical pencil using a cable entering into elongated housing through its cable end remote from the electrode blade end;

FIG. 7 shows an enlarged elevation view of the switch area of FIG. 6;

FIG. 8 shows a cross-sectional view taken along line A-A of FIG. 1;

FIG. 9 shows a side-sectioned elevation view of an electro-surgical pencil wherein the fiber optic switch is disposed partially in the longitudinally extending passageway of elongated tubular housing;

FIG. 10 shows a cross-sectional view taken along line B-B of FIG. 9;

FIG. 11 shows a diagrammatic representation of the fiber optic switch;

FIG. 12 is a schematic representation of the fiber optic switch showing the electrical cable and the optic fiber;

FIG. 13 is a perspective view of a ball and socket joint used to couple the surgical pencil to the smoke evacuation tube;

FIG. 14 is another perspective view of the ball and socket joint showing the interior thereof; and

FIG. 15 is an elevation view showing the surgical pencil with the ball and socket joint attached.

### DETAILED DESCRIPTION OF THE PREFERRED EMBODIMENTS

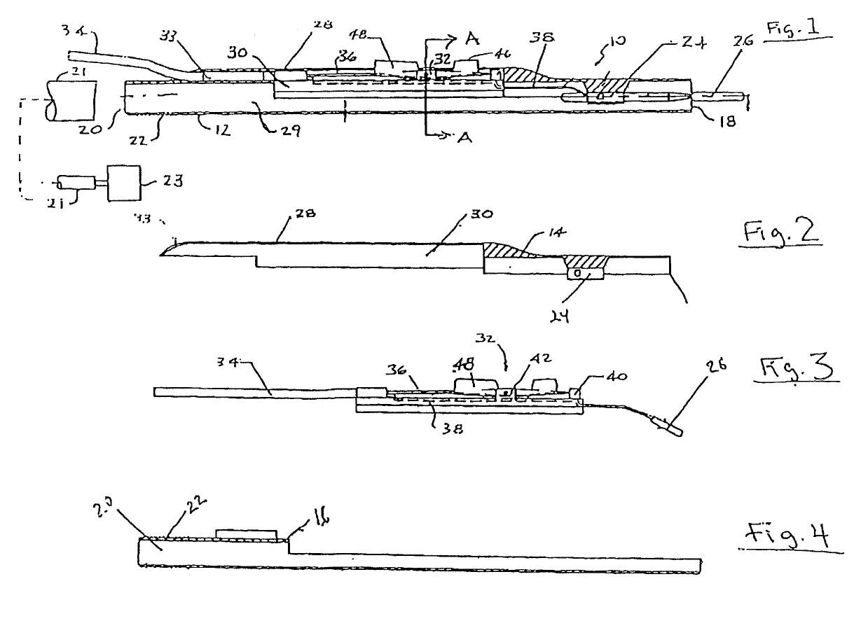

Referring now to FIG. 1, an electro-surgical pencil 10 has an elongated tubular housing 12, formed by a molding process. Elongated tubular housing 12 has a top half 14 (see FIG. 2) and a bottom half 16 (see FIG. 4), which when assembled define a cavity 30. Elongated tubular housing 12 is also provided with a substantially constant, circular cross-section having an inside diameter of approximately 8-mm, through which even bone fragments can pass. This cross-section is adequate to permit a flow and velocity of air sufficient to aspirate and evacuate the smoke produced by the electro-surgical pencil 10. Elongated tubular housing 12 is also characterized by a proximal extremity 18, directed toward a surgical site, and at an opposite end a distal extremity 20, the latter incorporating on its exterior a ribbed zone 22. Ribbed zone 22 is adapted for attachment to a vacuum tubing 21 to be connected with an independent vacuum source unit 23. An example of such a vacuum unit is the Buffalo, Plume Safety® Whisper™, which has a suction capacity of 35 cubic feet per minute at ⅞ inch diameter tubing.

When no smoke evacuation is intended, elongated tubular housing 12 can be designed without ribbed zone 22. Ribbed zone 22 is also unnecessary when the interior diameter of the vacuum tubing 21 is matched with the exterior diameter of elongated tubular housing 12 so that a reliable connection can be achieved. Top half 14 (see FIG. 2) of elongated tubular housing 12 is provided in its interior, close to proximal extremity 18, with a support 24. An electrode blade 26 is attached to support 24 and extends outside proximal extremity 18. An example of such an electrode blade 26 is the one sold by Aaron Medical under the trademark ESOI. Electro-surgical pencil 10 is designed to use electrode blades 26 compatible with standard radio frequency power supplies.

Elongated tubular housing 12 has a housing portion 28 enclosing a cavity 30. A fiber optic switch 32 is positioned within cavity 30.

In the present embodiment, housing portion 28 is provided with an entrance opening 33 for a cable 34 brought from the exterior of elongated tubular housing 12. Cable 34 carries an optic fiber 36 and an electrical conductor 38. Optic fiber 36 is connected to fiber optic switch 32, while electrical conductor 38 is attached to electrode blade 26.

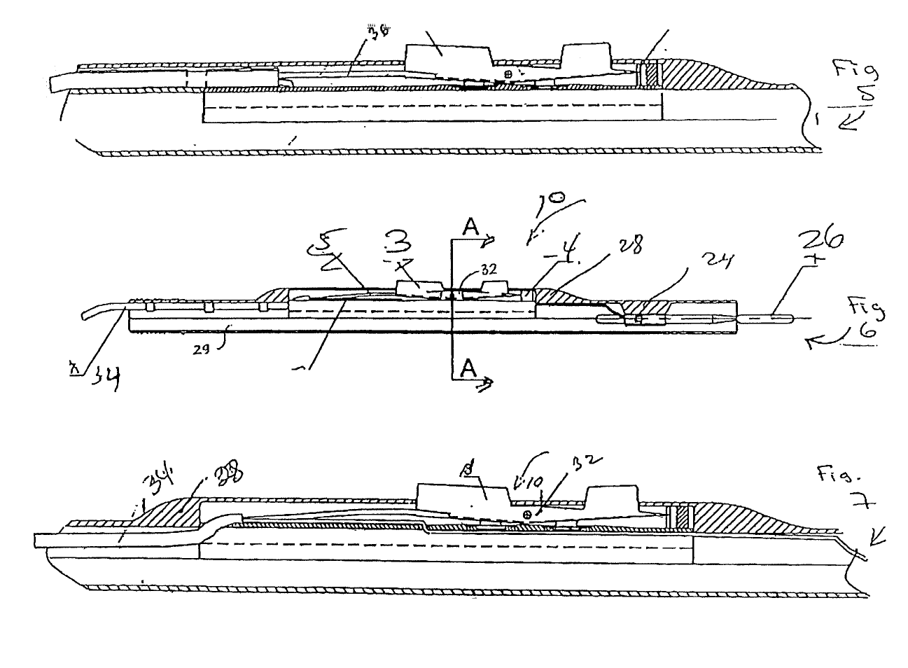

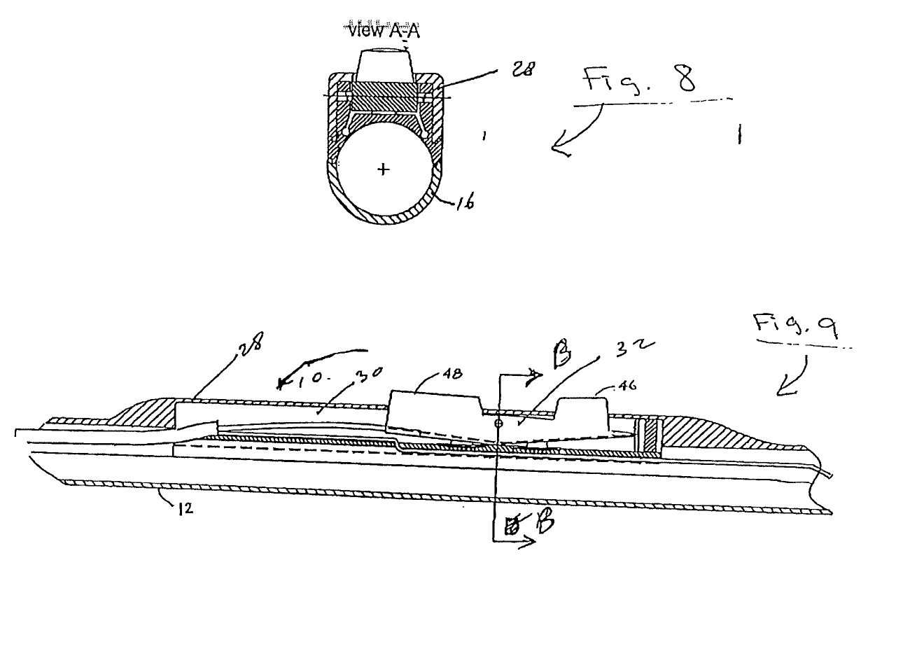

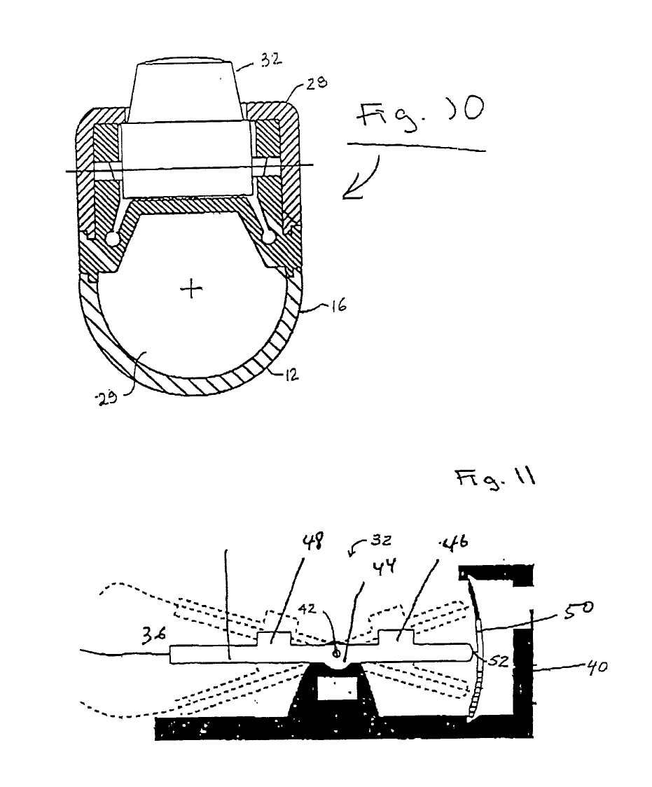

Alternately, in another embodiment shown in FIGS. 6 and 7, cable 34 instead of being brought from the exterior, as in the embodiment of FIGS. 1 to 5, enters into elongated tubular housing 12 through distal extremity 20. Referring to FIGS. 1 and 6, housing portion 28 is so configured, that fiber optic switch 32 is disposed outside the longitudinally extending passageway 29 formed in tubular housing 12. In this case, only electrode blade 26 and its support 24 constitute an obstruction to the smoke flow.

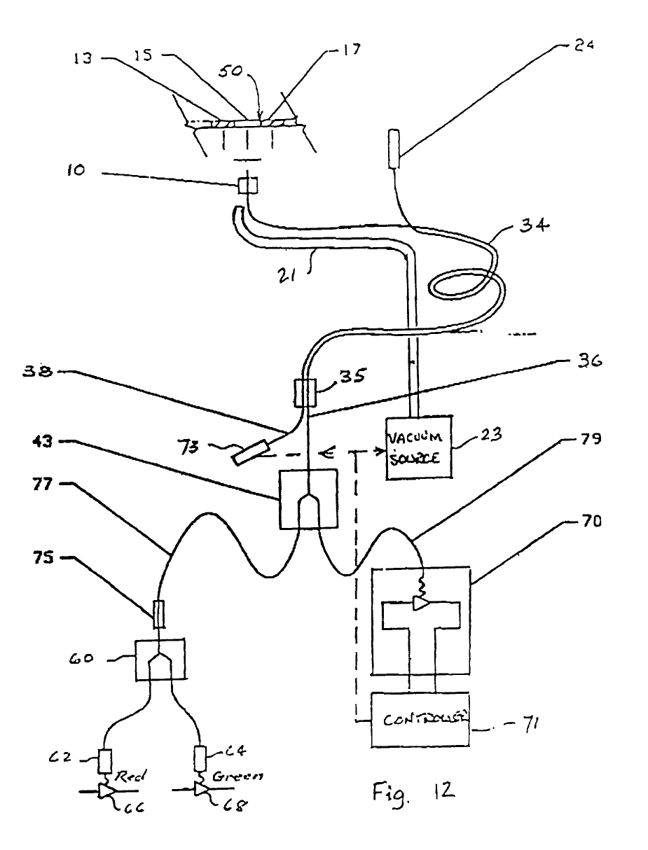

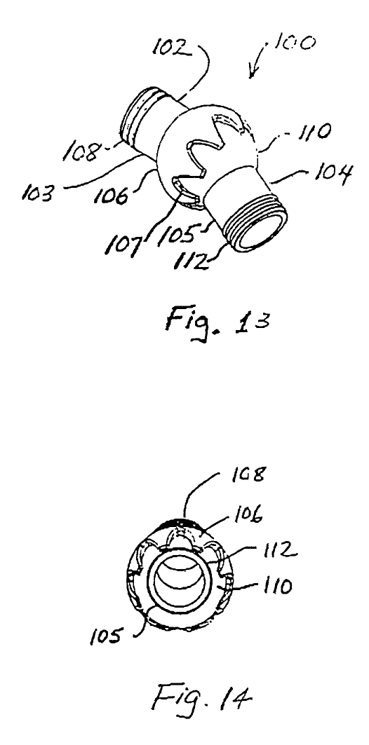

In another embodiment illustrated in FIGS. 9 and 10, fiber optic switch 32 disposed partially in the longitudinal passageway of elongated tubular housing 12. In this case, fiber optical switch 32 penetrates and occupies a space representing approximately 15-20% of the circular cross-section of elongated tubular housing 12. As can be seen, fiber optic switch 32 may be either partially internal or completely external to elongated tubular housing 12. Fiber optic switch 32 used in the present invention and illustrated diagrammatically in FIG. 11 is based on the type of switches disclosed in U.S. Pat. No. 5,046,806, granted to Kidder and in U.S. Pat. No. 5,892,862, also granted to Kidder both of which are incorporated herein by reference.

Fiber optic switch 32 (see FIG. 11) comprises a casing 40 to which a pivot 42 is affixed. An actuator 44 is reversibly movable between at least two switch positions and, therefore, is provided by a cutting button 46 and a coagulation button 48.

A flexible filter-mirror 50, also affixed to casing 40, is provided with 3 zones, namely, red, green and clear.

Optic fiber 36, which is a 250 μm polymethylmethacrylate, enters into actuator 44 at one end and exits at the opposite end with a tip 52 which contacts flexible filter-mirror 50. Light traveling along optic fiber 36 exits through tip 52 and is reflected back into optic fiber 36 by either of two colors or the clear zone of flexible filter-mirror 50. Light traveling along optic fiber 35 exits through tip 52 and is reflected back into optic fiber 36 by either of two colors or the clear zone of flexible filter-mirror 50, depending on which one of cutting button 46 or coagulation button 48 is depressed.

An opto-electronic adapter (not shown) determines which button is depressed by distinguishing which color of flexible filter-mirror 50 is opposite tip 52. This is done by launching alternately RED then YELLOW or GREEN light into optic fiber 36 towards proximal extremity 18 (see FIG. 1) and establishing the ratio of the amplitude of the reflected RED to YELLOW or RED to GREEN light. The RED and GREEN colored portions of flexible filter-mirror 50 attenuate YELLOW or GREEN and RED lights, respectively.

If RED light is reflected 50% greater than YELLOW, then tip 52 rests on the RED zone of flexible filter-mirror 50. If YELLOW or GREEN light is reflected 50% greater than RED, then tip 52 rests on the YELLOW or GREEN zone. If the ratio of the two returning light levels are within +/−49%, then tip 52 does not rest completely on either RED zone or on the YELLOW or GREEN zone. Fiber optic switch 32 is selectively electrically connectable to an external power source. Thus, by depressing cutting button 46 of fiber optic switch 32, electrode blade 26 is electrically connected to the power source and heated adequately for incising anatomical structures. Alternately, by depressing coagulation button 48, electrode blade 26 (see FIG. 1) is electrically connected to the power source and delivers energy to the tissue to perform coagulation of bleeding blood vessels. Movement of actuator 44 into the cutting or coagulation positions can, when conventional circuit connections are made, automatically connect the interior of elongated tubular housing 12 (see FIG. 1) with a vacuum source unit 23 (see FIG. 1). Thus, the interior of elongated tubular housing 12 becomes a passageway for smoke evacuation.

Referring to FIG. 12 the electro-surgical pencil 10 is coupled by cable 34 to cable holder 35 where the electrical conductor 38 is separated from the optic fiber 36. The optic fiber 36 enters beam splitter 43 while the electrical conductor 38 couples to a source of RF power 73. Light fiber 77 couples through ferrule 75 to beam splitter 60 to which is coupled a source of red light 66 through ferrule 62 and a source of green (or yellow) light 68 through ferrule 64. Reflected light travels along fiber 79 to a light detector 70 where the intensity of the reflected light is measured. The intensity measurement is fed to a controller 71, which computes the intensity ratio of successive light pulses. If the RED to YELLOW or GREEN intensity ratio is 1.5 then the tip 52 is opposite the RED region of the flexible filter-mirror 50. If the ratio is 0.5 then the tip 52 is opposite the YELLOW or GREEN region. Controller 71 computes this ratio and sends a signal to the source of RF power 73, which sends sufficient power up electrical conductor 38 to incise anatomical features if the ratio is 1.5. If the ratio is 0.5, the source of RF power 73 sends just enough power up electrical conductor 38 to perform coagulation of bleeding blood vessels. The same signal from the controller 71 is applied to the vacuum source unit 23 and activates it whenever the source of RF power 73 is activated so as to draw a vacuum through vacuum tubing 21.

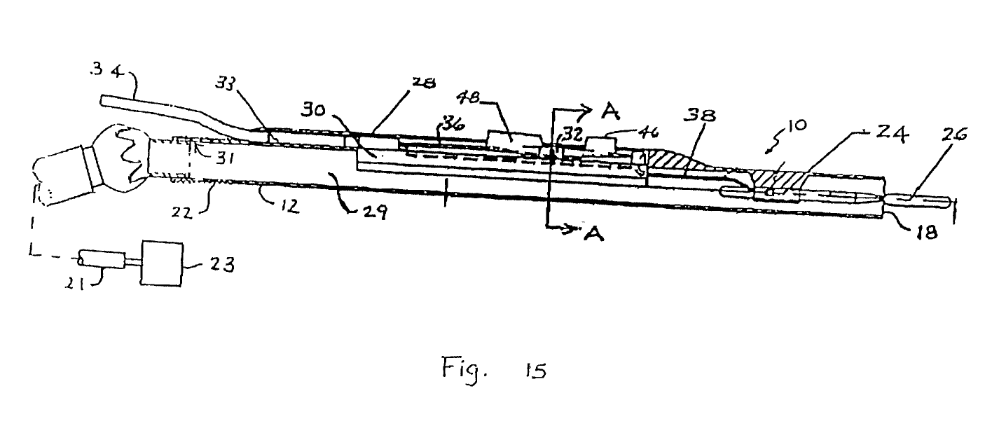

Referring to FIGS. 13 and 14 the ball and socket joint 100 having a socket 102 and ball 104. The socket 102 has open hemispherical female shell 106 with regularly spaced indents 107 around its periphery. The indents 107 give the open shell flexibility so that it may permit ball 110 to be removably inserted. A series of circular ribs 108 extend around a round tubular portion 103 and function to retain a vacuum tubing 21 (see FIG. 15) fitted over the ribs 108. The ball 104 also has a tubular end 105 having a threaded end 112 for threaded reception by a corresponding threaded end 31 (see FIG. 15) of the elongated tubular housing 12. The ball 104 and socket 102 are made of plastic. The large surface area of the ball 104 and socket 102 allow a swiveling movement of the two during evacuation without losing the vacuum seal.

Accordingly, while this invention has been described with reference to illustrative embodiments, this description is not intended to be construed in a limiting sense. Various modifications of the illustrative embodiments, as well as other embodiments of the invention, will be apparent to persons skilled in the art upon reference to this description. It is therefore contemplated that the appended claims will cover any such modifications or embodiments as fall within the true scope of the invention.

What is claimed is:

1. An electro-surgical pencil of a type having an electrode at a proximate extremity of said pencil, an electrical conductor coupled to said electrode and operative to couple said electrode to an external source of electrical power, comprising:
(a) an elongated housing having a hollow interior passageway, and a distal extremity opposite to said proximate extremity couplable to a vacuum source and wherein said hollow interior passageway is of a diameter sufficiently large to permit a flow and velocity of air sufficient to aspirate and evacuate smoke produced by the pencil;
(b) an electrode support in the hollow interior passageway which holds said electrode and wherein said electrode support and said electrode present a cross-sectional area which is substantially less than that of said hollow interior passageway; and
(c) an optical switch coupled to said elongated housing operative to switch power to said electrode in response to movement of said optical switch to a selected switch position.

2. The electro-surgical pencil according to claim 1, including:
(a) an optical target having at least two different types of surfaces, said optical target coupled to said elongated housing proximate said optical switch; and
(b) a light fiber coupled to said switch, an end of said light fiber proximate said target, said end positionable to direct light selectably onto one of the two different types of surfaces and to collect light given off by said one surface and direct it back up the light fiber.

3. The pencil of claim 1, including a switch sensor and a beam splitter coupled to said light fiber operative to separate reflected light from transmitted light and to direct said reflected light to the switch position sensor.

4. The pencil of claim 2, wherein said vacuum source is coupled to said switch position sensor and is operative to become activated in response to said switch sensor detecting said optical switch being switched to activate said electrode.

5. The pencil of claim 1, wherein said hollow interior passageway is circular.

6. The pencil of claim 2, wherein the diameter of said hollow interior passageway is 8 mm.

7. The pencil of claim 1, wherein said switch occupies less than 20% of the cross sectional area of said hollow interior passageway.

8. The pencil of claim 1, wherein said light fiber extends along said hollow interior passageway and through an external hollow coupling to the vacuum source.

9. The pencil of claim 1, wherein the cross sectional dimensions of said hollow interior passageway are constant.

10. The pencil of claim 1, wherein said housing is made of a pair of longitudinally split halves which define a recess there between.

11. The pencil of claim 1, wherein said optical target is a flexible filter-mirror having a reflective surface covered by two different filters each with a different colour overlying said reflective surface.

12. The pencil of claim 11, wherein said filters are incorporated into a flexible film.

13. An electro-surgical pencil of a type having an electrode at a proximate extremity of said pencil, an electrical conductor coupled to said electrode and operative to couple said electrode to an external source of electrical power, comprising:
(a) an elongated housing having a hollow interior passageway, and a distal extremity opposite to said proximate extremity couplable to a vacuum source and wherein said hollow interior passageway is of a diameter sufficiently large to permit a flow and velocity of air sufficient to aspirate and evacuate smoke produced by the pencil;
(b) an optical switch coupled to said elongated housing operative to switch power to said electrode in response to movement of said optical switch to a selected switch position; and
(c) a ball socket joint coupled to said distal extremity.

14. The electro-surgical pencil according to claim 13, including an electrode support in the hollow interior passageway operative to hold said electrode and wherein said electrode support and said electrode present a cross-sectional area which is substantially less than that of said hollow interior passageway.

15. The electro-surgical pencil according to claim 13, wherein said ball and socket joint has a ball and a socket, said socket having indents around its periphery to provide flexibility for insertion of said ball.

16. The electro-surgical pencil according to claim 15, wherein said ball and socket are each shells having a hollow interior.

17. The electro-surgical pencil according to claim 15, wherein said ball and socket have a tubular portion affixed to said ball and said socket, respectively, and a diameter of each of said ball and said socket are larger than a diameter of their respective tubular portions.

18. The electro-surgical pencil according to claim 17, wherein the tubular portion of one of said ball and socket have spaced apart circumferential ribs operative to grip a tube inserted thereover.
